(19) United States
(12) Patent Application Publication
(10) Pub. No.: US 2008/0039760 A1
(43) Pub. Date: Feb. 14, 2008

(54) Removable gauze bandage with perforated adhesive strips to adjust bandage

(76) Inventor: Joseph John Lesko, Huntington Beach, CA (US)

Correspondence Address: STETINA BRUNDA GARRED & BRUCKER, 75 ENTERPRISE, SUITE 250, ALISO VIEJO, CA, 92656, US

(21) Appl. No.: 11/503,760

(22) Filed: Aug. 14, 2006

(51) Int. Cl. A61F 13/00

(52) U.S. Cl. 602/42

(57) ABSTRACT

An adjustable adhesive bandage, comprising a flexible planar surface. The flexible planar surface includes an adhesive side, a non-adhesive side, a peripheral edge, a long axis and a short axis. The adhesive bandage has a gauze pad that is peelably engaged to the adhesive side of the flexible planar surface. A plurality of perforations are emplaced across the flexible planar surface. The plurality of perforations may be lacerated to adjust the shape or size of the adhesive bandage. The adhesive bandage may further comprise a waterproof cover peelably engaged to the flexible planar surface. The adjustable adhesive bandage may be laminated to germ proof and waterproof the bandage.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The present invention relates to adhesive bandages and, in particular, a uniquely adjustable bandage with a removable gauze pad. The gauze pad may be removed from the adhesive bandage to clean the wound. The adhesive bandage may be adjusted to effectively dress the wound.

A sticking plaster also commonly known as a band-aid is a small adhesive bandage used for relatively minor injuries. The plaster usually consists of an absorbent pad often medicated with antiseptic covered by a layer of woven fabric or of plastic, which is covered in a layer of adhesive. The plaster is applied such that the absorbent pad covers the wound, and the fabric or plastic sticks to the surrounding skin to hold the dressing in place and prevent bacteria from entering the wound. Although the common household sticking plaster has been used to treat minor wounds for a long time, there remain various deficiencies with its use. The common household sticking plaster is often too small to dress a wound effectively. Additionally, there are times when it is necessary to prevent water from entering the wound. Typically, the common household sticking plaster is not waterproof. The fabric or plastic that is supposed to stick to the surrounding skin to hold the dressing in place normally fails to stick or sticks to the skin for a short period. The common household sticking plaster is even more ineffective on curved or moving areas such as ones knees, knuckles or elbows where the fabric or plastic cannot be applied evenly. A ripple underneath the sticking plaster will usually appear which prevents the absorbent pad from effectively dressing the wound. Often, the sticking plaster will fall off immediately after application to a wound located on a curved or moving area. Another deficiency in the common household sticking plaster is that it is not 100% germ proof. This is very important, because a sticking plaster that is germ proof can prevent infection in the wound. Additionally, the common household sticking plaster cannot be used to clean a cut, scratch, or wound. Typically, the wound is cleaned before the sticking plaster is applied.

In order to perform all of the above-mentioned tasks, one must stock their medicine chest or cabinet like a first-aid station in a large manufacturing plant. Various types of bandages would have to be included in the first-aid station to treat different size wounds, curved areas or moving areas.

Currently, there isn't a single box of bandages on the retail shelf at the local grocery store or drug store that fulfills the need for different types of bandages incorporated into a single bandage.

As can be seen, there exists a need in the art for an adjustable bandage that provides proper protection for various types of wounds and can also be used to clean the wound.

### BRIEF SUMMARY OF THE INVENTION

It is therefore an object of this invention to provide a household bandage that will perform all the above tasks. The present invention specifically addresses and alleviates the above-referenced deficiencies associated with the common household sticking plaster. More specifically, the present invention has a removable gauze pad and is adjustable in size or adjustable in shape.

In its broadest sense, the adjustable adhesive bandage includes a flexible planar surface. The planar surface includes an adhesive side, a non-adhesive side, a peripheral edge, a long axis and a short axis. The adhesive side of the planar surface sticks to the skin surrounding the wound to secure an antiseptic gauze pad, absorbent pad or cotton ball to the wound. The peripheral edge forms the boundary of the adjustable adhesive bandage. The adjustable adhesive bandage includes a removable gauze pad peelably engaged to the adhesive side of the flexible planar surface. It is contemplated that the gauze pad is peelably engaged to the planar surface using an adhesive such as the adhesive used on the back of a post-it note produced by 3M. The advantage of removing the gauze pad includes the ability of cleaning the wound by applying an antiseptic to the pad before the adhesive bandage is applied to the wound. Otherwise, the gauze pad may remain engaged to the adhesive bandage and the adhesive bandage may be applied to the wound similar to a common household sticking plaster. The adjustable adhesive bandage includes a plurality of perforations emplaced across the flexible planar surface. The plurality of perforations on the flexible planar surface allows the user of the adhesive bandage to adjust the size or shape of the bandage. The adjustments are made by lacerating the perforations as discussed in more detail below.

Another embodiment of the invention includes a plurality of perforations formed across the long axis of the flexible planar surface. Thus, if the perforations are lacerated across the long axis of the flexible planar surface the adjustable adhesive bandage forms a butterfly bandage. A butterfly bandage consists of four flaps and is more effective as a bandage for curved areas or areas with a lot of movement such as various joints.

In yet another embodiment of the invention includes a plurality of perforations formed across the short axis of the flexible planar surface. Thus, if the perforations are lacerated across the short axis of the planar surface the adjustable adhesive bandage forms a smaller bandage. A smaller bandage may be preferred for smaller wounds or wounds where the flexible planar surface of the bandage is more effective in a smaller or shorter form.

Another embodiment contemplates perforations across the long and short axis of the flexible planar surface. The emplaced perforations form removable sections of the flexible planar surface. This type of adjustable adhesive bandage has many advantages. Perforations across both the long and short axis allow for more combinations of shapes and sizes for adjustment. The bandage can be adjusted to form a butterfly bandage. If a butterfly bandage is not desired, the size of the bandage may be reduced.

The adjustable bandage further comprises a removable gauze pad. The removable gauze pad is peelably engaged to a portion of the adhesive side of the planar surface by a lift-off strip. The lift-off strip includes finger tabs at opposite ends. The finger tabs of the lift off strip are specially coated to prevent the finger tabs from sticking to the adhesive side of the flexible planar surface. Thus, to remove the gauze pad, the user simply lifts the lift-off strip at the finger tab. The advantage of removing the gauze pad from the adhesive strip is that the user of the adjustable bandage can apply an antiseptic solution to the gauze pad such as alcohol, hydrogen peroxide or other germicides to wipe wound clean and to sterilize and treat the wound. After the wound is clean, the user of the adjustable bandage can place an absorbent pad on the wound and secure the pad using the adjustable adhesive bandage.

The adjustable bandage may further comprise a waterproof cover peelably engaged to the adhesive side of the flexible planar surface. When the adjustable adhesive bandage is applied over a wound, it may be desired to prevent water from entering the wound. To prevent water from entering the wound, the waterproof cover remains peelably engaged to the flexible planar surface of the bandage. If the waterproof cover is not desired, the cover may be lifted from the planar surface by lifting the waterproof cover tab. When the waterproof cover is removed, an air hole is revealed. The air hole portion of the adjustable adhesive bandage is placed over an absorbent pad, gauze pad, or cotton ball before securing the bandage by sticking the adhesive side of the flexible planar surface to the skin surrounding the wound.

In yet another embodiment, an adjustable bandage may further comprise a cotton ball peelably engaged to the adhesive side of the flexible planar surface. A cotton ball may be preferred when wiping the wound clean.

The adjustable bandage may further comprise an absorbent pad peelably engaged to the adhesive side of the flexible planar surface. The advantage of this embodiment is the ability to clean the wound using an absorbent pad that may be preferred over a cotton ball or gauze pad.

Additionally, the adjustable bandage may further comprise adjustable bandages overlapping, such that the perforations across the short axis of the flexible planar surfaces are substantially aligned across the peripheral edge of the planar surfaces. The bandages may also be laminated. There are a number of advantages of this embodiment of the invention. The overall size of the adjustable bandage is increased, thus it can be applied to larger wounds. Another practical advantage includes the many different combinations of adjustments that are possible by lacerating different perforations along the flexible planar surface of the adhesive bandage. Laminating the bandages provides a waterproof and germproof bandage. Therefore, the adjustable bandage encompasses a variety of different adhesive bandages in one package.

### BRIEF DESCRIPTION OF THE DRAWINGS

These, as well as other features of the present invention, will become more apparent upon reference to the drawings wherein:

FIG. 1 is a top view of the adjustable bandage.

FIGS. 2A, 2B, and 2C are side views illustrating a removable gauze pad.

FIG. 3 is a top view illustrating a butterfly bandage.

FIG. 4 is a top view illustrating a small bandage.

FIGS. 5A and 5B are top views illustrating a bandage with waterproof cover and vent hole.

FIG. 6 is a side view illustrating a cotton ball being removed from the adhesive side of a flexible planar surface.

FIG. 7 is a top view illustrating three bandages overlapping and laminated together.

FIG. 8 is a top view illustrating a lift-off strip.

FIGS. 9A, 9B, and 9C illustrate a wound being dressed with an adjustable bandage on a human finger.

FIG. 10 is a top view illustrating a butterfly type bandage with a large air hole.

FIGS. 11A, 11B, and 11C are side views illustrating a bandage with absorbent sponge being removed.

### DETAILED DESCRIPTION OF THE INVENTION

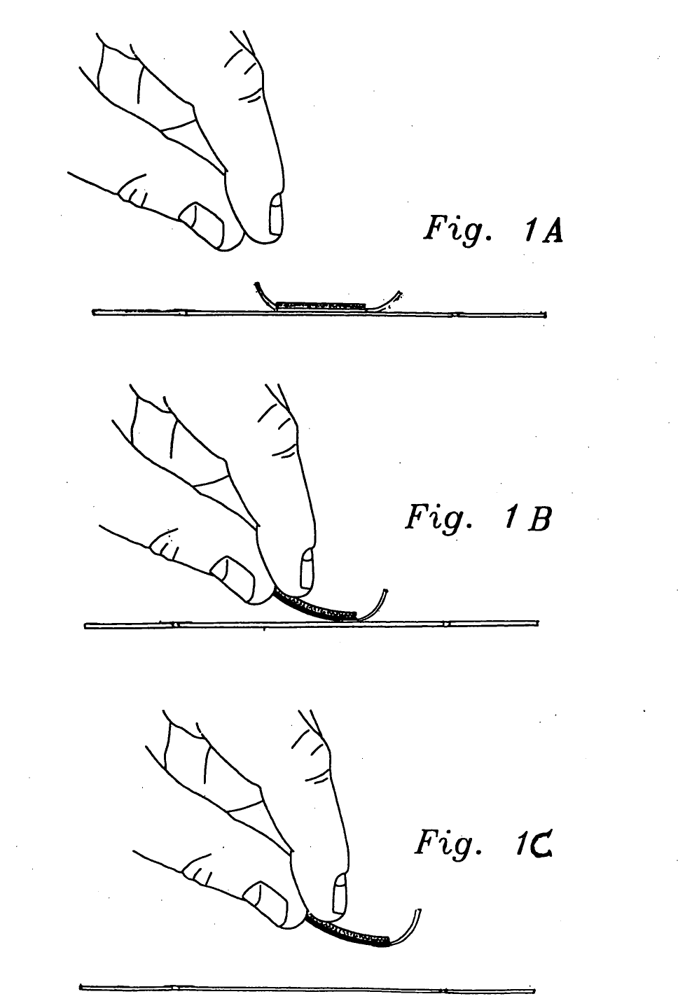

Referring now to the drawings wherein the showings are for purposes of illustrating preferred embodiments of the present invention only and not for purposes of limiting the same, shown in FIG. 1 is an adjustable adhesive bandage 10. The bandage 10 includes a flexible planar surface 12 having an adhesive side 14 and a non-adhesive side 16. The bandage also includes a peripheral edge 18. Additionally, the flexible planar surface includes a long axis 20 and a short axis 21 across the planar surface 12. A gauze pad 22 peelably engaged to a portion of the adhesive side 14 of the planar surface 12. A plurality of perforations emplaced across the flexible planar surface. The peripheral edge 18 of the planar surface 12 defines the boundary of the adjustable adhesive bandage 10. The adhesive side 14 of the flexible planar surface sticks to the skin surrounding the wound in order to keep various types of antiseptic pads secured to the wound. The perforations 24, 26 emplaced on the flexible planar surface allow for adjustments in the bandage 10. The adjustments are made by lacerating the perforations 24, 26.

The adjustable adhesive bandage 10 may further comprise a plurality of perforations 24 across the long axis 20 of the planar surface 12. Furthermore, the adhesive bandage 10 may comprise of a plurality of perforations 26 across the short axis 21. The plurality of perforations 24, 26 across the short axis 21 and the long axis 20 form sections of flexible planar surface 12 that are capable of being removed to adjust the size or shape of the adhesive bandage 10.

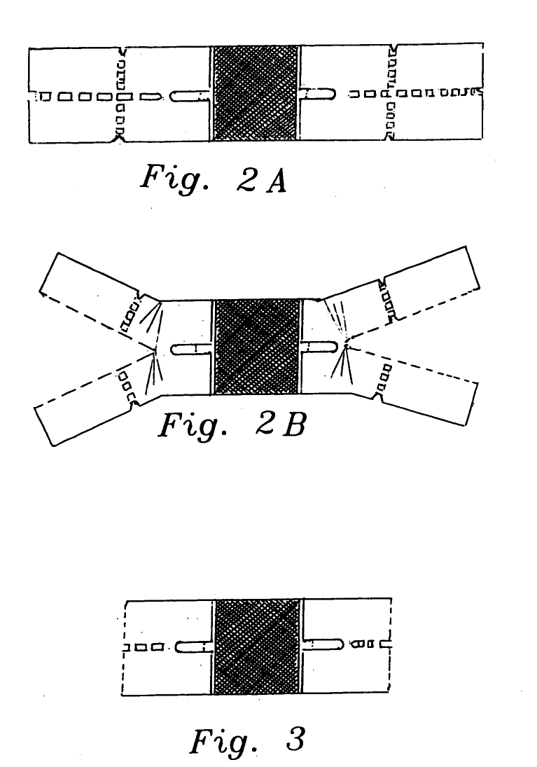

Referring now to FIG. 2 A-C, shown is a side view of the flexible planar surface 12. The removable gauze pad 22 is peelably engaged to a portion of the adhesive side 14 of the planar surface by a lift-off strip 30. The removable gauze pad 22 is secured to the lift-off strip 30 by an adhesive 34. The lift-off strip 30 includes finger tabs 32 at opposite ends. The finger tabs 32 are specially coated so the lift-off strip 30 will not stick to the adhesive side 14 of the planar surface 12 where the finger tabs 32 are located. The gauze pad 22 is removed by lifting the lift-off strip 30 using the finger tabs 32. When the gauze pad 22 is removed, antiseptic such as alcohol or hydrogen peroxide may be applied to the gauze pad 22 to clean the wound. The embodiment of the invention contemplates using the removable gauze pad 22 to destroy, prevent, or inhibit growth of bacteria in and around the wound. Thus, the adjustable adhesive bandage 10 may be used to clean the wound rather than dress the wound. In another embodiment, the adjustable adhesive bandage 10 may be used to clean the wound and to dress the wound.

Referring now to FIG. 3, shown is a butterfly bandage 28 formed from the adjustable bandage 10. The butterfly bandage 28 is formed by lacerating the perforations 24 along the long axis 20 of the flexible planar surface 12. The advantage of the butterfly bandage 28 is the four flaps that allow the adhesive bandage to remain secure on a curved or moving area. The common household bandage is generally ineffective in securing the absorbent pad to the skin over a curved or moving area.

FIG. 4 illustrates a small bandage 36. The small bandage 36 is formed from the larger adjustable bandage 10. The small bandage 36 is formed by lacerating the perforations 26 along the short axis 21 of the flexible planar surface 12. A section of the flexible planar surface 12 is removed to form the smaller bandage 36. A smaller adhesive bandage 36 is preferred for smaller wounds.

Referring now to FIGS. 5A and 5B, shown is an adjustable bandage 10, further comprising a waterproof cover 38 peelably engaged to the flexible planar surface 12. The waterproof cover 38 prevents water from entering the wound. If waterproofing the wound is not desired, the vent cover 38 may be removed by lifting the waterproof cover tab 42. When the vent cover 38 is removed, the air hole 40 is exposed. This embodiment of the invention has the advantage of securing an absorbent pad to a wound with the ability to decide whether to waterproof the absorbent pad or keep the absorbent pad unprotected from water by placing the air hole 40 of the adhesive bandage over the absorbent pad.

Referring now to FIG. 6, a cotton ball 44 is secured to the removable gauze pad 22. The cotton ball 44 may be preferred as an agent to clean wound or apply an anti-bacterial treatment. The gauze pad 22 including the cotton ball 44 may be removed from the adhesive side 14 of the flexible planar surface 12.

FIG. 7 illustrates three adjustable bandages 10 overlapped such that the perforations 26 across the short axis 21 of the flexible planar surface 12 are substantially aligned at the peripheral edge 18. The bandages 10 are laminated together to form a large adjustable bandage 46 that is both waterproof and germ proof. The large adjustable bandage 46 can be adjusted to form many different types of shapes and sizes. The larger size bandage may be desirable for larger wounds where the size of a common household bandage is inadequate. Furthermore, the larger adjustable adhesive bandage 46 has many emplaced perforations allowing for a multitude of adjustments in shape and size.

FIG. 8 shows the lift-off strip 30 and the finger tab 32 with a cotton ball 44 secured to the lift-off strip 30.

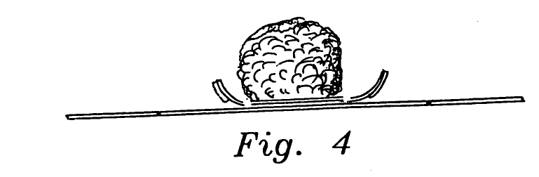

Referring now to FIGS. 9 A-C, illustrates the adjustable bandage 10 applied to a wounded fingertip. The removable gauze pad 22, absorbent pad, or antiseptic pad is applied to the wound. Then the adjustable bandage 10 is applied around the finger, securing the gauze pad 22, absorbent pad, or antiseptic pad to the wound. The embodiment of the invention as shown illustrates the advantage of removing the gauze pad 22 and securing the gauze pad 22 to the injury using the adjustable bandage 10. Thereby, the adjustable adhesive bandage 10 is used to clean the wound and then to dress the wound.

FIG. 10 illustrates a butterfly bandage 28. The butterfly bandage 28 having a large air hole 48. A gauze pad 22 or a cotton ball 44 is placed on the injury and the large air hole 48 is placed over the gauze pad 22 or cotton ball 44 to secure the bandage 28 on the wound.

FIGS. 11 A-C illustrate an absorbent pad 50 being removed from the adhesive side 14 of the flexible planar surface 12.

1. An adjustable adhesive bandage, comprising:
a flexible planar surface having an adhesive side, a non-adhesive side, a peripheral edge, a long axis and a short axis;
a non-adhesive pad peelably engaged to the adhesive side of the flexible planar surface;
a plurality of perforations emplaced across the flexible planar surface.

2. The bandage of claim 1, further comprising a plurality of perforations across the long axis of the flexible planar surface.

3. The bandage of claim 2, further comprising a plurality of perforations across the short axis of the flexible planar surface.

4. The bandage of claim 3, wherein the plurality of perforations across the long axis intersect the plurality of perforations across the short axis.

5. The bandage of claim 1, further comprising a waterproof cover peelably engaged to the adhesive side of the flexible planar surface.

6. The bandage of claim 1, wherein the non-adhesive pad is a gauze pad peelably engaged to the adhesive side of the flexible planar surface.

7. The bandage of claim 1, wherein the non-adhesive pad is an absorbent pad peelably engaged to the adhesive side of the flexible planar surface.

8. The bandage of claim 1, further comprising adjustable bandages overlapping, such that the perforations across the short axis of the flexible planar surfaces are substantially aligned across the peripheral edge of the planar surfaces.

9. The bandage of claim 8, wherein the overlapping bandages are laminated.

10. The bandage of claim 1, wherein the non-adhesive pad is a cotton ball peelably engaged to the adhesive side of the flexible planar surface.
